(19) United States
(12) Patent Application Publication
(10) Pub. No.: US 2024/0091497 A1
(43) Pub. Date: Mar. 21, 2024

(54) CURVED S-SHAPE FOR CATHETER SHAFT AND DISTAL TIP

(71) Applicant: TERUMO KABUSHIKI KAISHA, Tokyo (JP)

(72) Inventors: Yuichi TADA, Santa Clara, CA (US); John BARRITT, San Jose, CA (US); Mike FOSTER, East Palo Alto, CA (US)

(21) Appl. No.: 17/946,963

(22) Filed: Sep. 16, 2022

(51) Int. Cl. A61M 25/00; A61M 25/09

(52) U.S. Cl. CPC A61M 25/0068; A61M 25/0021; A61M 25/09

(57) ABSTRACT

A catheter for removing an object in a body lumen includes an outer tube, a drive shaft surrounded by the tube, a cutting member connected to a distal end of the shaft to be rotated by the shaft with respect to a rotation axis and by which the object is cut, a guide wire tube attached to a distal end of the outer tube and having a first lumen extending adjacent to the cutting member along the axis, and a distal tip attached to a distal end of the guide wire tube and having a second lumen extending parallel to the rotation axis and communicating with the first lumen. The drive shaft includes a curved portion near the distal end thereof to cause the tip to contact a first inner surface of the body lumen when the curved portion contacts an opposite second inner surface of the body lumen.

### TECHNICAL FIELD

Embodiments of the present disclosure relate generally to a catheter, a medical device, and a method for removing an object in a body lumen.

### BACKGROUND

Medical devices that have a catheter including a rotatable drive shaft and a cutting member are widely used to remove an object from a body lumen such as a blood vessel. Such medical devices have a motor for generating torque and a hub for storing a mechanism which transmits the generated torque to the cutting member through the drive shaft.

A catheter has at its distal end a guide wire lumen into which a guide wire for guiding the catheter inside a body lumen is inserted. At the beginning of an operation, an introducer sheath is inserted into the body vessel, and then the guide wire is inserted through the sheath toward and beyond a target object to be removed. Subsequently, the guide wire outside the body is inserted into the guide wire lumen of the catheter, which is then inserted through the introducer sheath into the body lumen along the guide wire. Once the distal end of the catheter has reached the object, the motor is turned on causing the cutting member to rotate, and the catheter is further moved forward so that the cutting member contacts and cuts the object.

A conventional standardized catheter has a cutting diameter of up to 2 mm and thus can effectively cut an object that is present in a body lumen of up to 2 mm. However, using the conventional standardized catheter, it is difficult to effectively cut an object that is present in a body lumen of a larger diameter, e.g., 4 mm.

### SUMMARY OF THE INVENTION

In an embodiment, a catheter for removing an object in a body lumen includes an outer tube, a rotatable drive shaft surrounded by the outer tube, a cutting member connected to a distal end of the drive shaft to be rotated by the drive shaft with respect to a rotation axis and by which the object is cut, a guide wire tube attached to a distal end of the outer tube and having a first guide wire lumen extending adjacent to the cutting member along the rotation axis, and a distal tip attached to a distal end of the guide wire tube and having a second guide wire lumen extending parallel to the rotation axis and communicating with the first guide wire lumen. The drive shaft includes a first curved portion near the distal end of the drive shaft to cause the distal tip to contact a first inner surface of the body lumen when the first curved portion contacts a second inner surface of the body lumen that is on an opposite side of the body lumen with respect to the first inner surface.

### BRIEF DESCRIPTION OF THE DRAWINGS

FIGS. 1 and 2 depict a medical device including a catheter and a handle in one embodiment.

FIGS. 3 and 4 depict a distal portion of the catheter.

FIG. 5 depicts a proximal portion of the catheter and a hub.

FIG. 6 depicts a curved portion of the catheter.

FIGS. 7A and 7B depict the catheter of FIG. 6 guided along a guide wire in a body lumen.

FIGS. 8 and 9 depict a catheter in one embodiment.

FIGS. 10 and 11 depict a catheter with two curved portions in one embodiment.

FIG. 12 depicts the catheter of FIG. 10 guided along a guide wire in a body lumen.

FIGS. 13A and 13B depict the catheter of FIG. 10 guided along a guide wire in a body lumen.

FIG. 14 depicts a flowchart of a method for removing an object in a body lumen.

### DESCRIPTION OF EMBODIMENTS

The following detailed description describes a catheter, a medical device, and a method for cutting an object inside a body lumen. In the present specification, a side of the medical device or the catheter which is inserted into a body lumen is defined as a distal side, and the other side of the medical device which is held by an operator during an operation is defined as a proximal side.

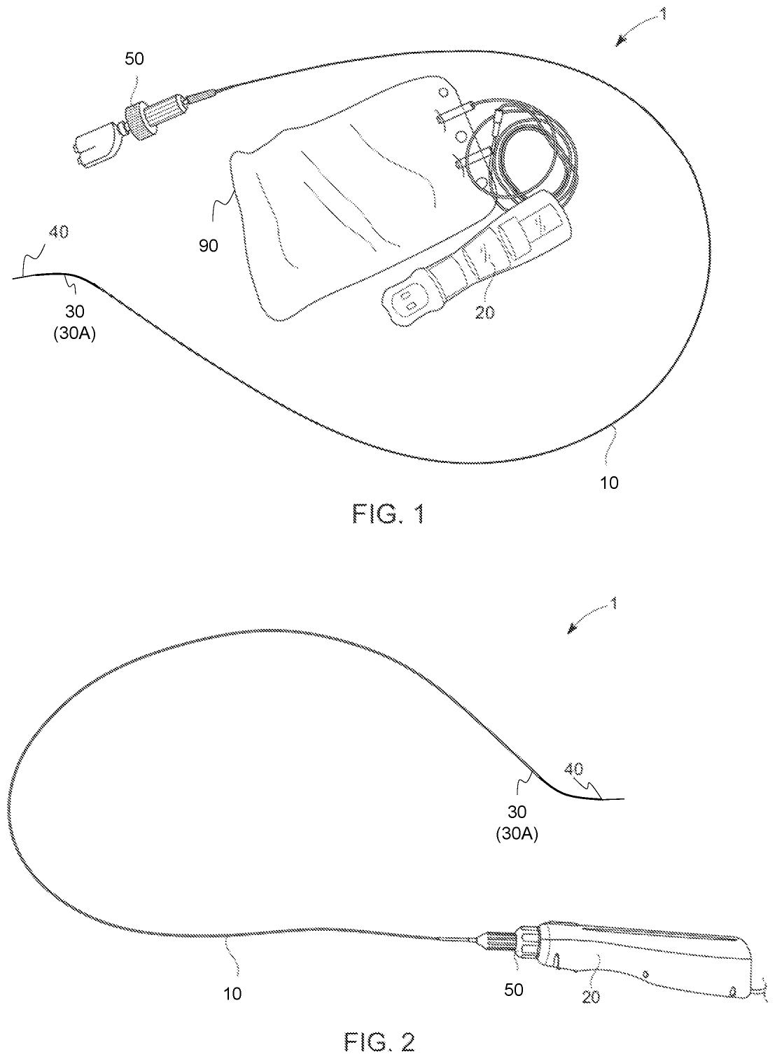

FIGS. 1 and 2 depict a medical device 1 in one embodiment. As shown in the figures, the medical device 1 includes a catheter 10 to be inserted into a body lumen for removing an object and a handle 20 held by an operator. The handle 20 includes a torque generating element such as a motor and an aspiration pump to aspirate the object that has been removed from the body lumen. FIG. 1 shows a state of the medical device 1 in which the catheter 10 is detached from the handle 20, and FIG. 2 shows a state in which the catheter 10 is attached to the handle 20.

The catheter 10 includes an elongated drive shaft 30 surrounded by an outer tube 30A and rotatable therein, and further includes, at its distal end, a cutting member 40 that rotates together with the drive shaft 30 to cut an object. For example, the cutting member 40 is a directional cutter for removing an object located in a particular direction. At the proximal end of the catheter 10, a hub 50 for housing rotating and aspiration mechanisms is attached. The hub 50 can be integrated into the catheter 10 or detachable from the catheter 10.

The drive shaft 30 has the characteristics of being flexible and capable of transmitting rotational power applied from the proximal side to the distal side. Specifically, the drive shaft 30 transmits the rotational torque generated by the torque generating element to the cutting member 40. The drive shaft 30 has an aspiration lumen through which the object that has been cut by the cutting member 40 is moved to the proximal side. The aspirated object is stored in a collection bag 90. The drive shaft 30 penetrates the outer tube 30A, and the cutting member 40 is fixed to the distal end of the drive shaft 30. The proximal portion of the drive shaft 30 is positioned inside of the hub 50.

The drive shaft 30 has a distal opening at which the aspiration lumen opens at the distal end thereof. The distal opening is an entrance into which the cut object enters. The proximal end of the drive shaft 30 is connected to an aspiration port through which the object that has entered the drive shaft 30 is discharged.

In one embodiment, a distal portion of the drive shaft 30 is curved to form an angle between the rotation axis of the cutting member 40 and the other portion of the drive shaft 30 as shown in FIGS. 1 and 2. With this angle, when the distal end of the catheter 10 is guided to a particular area of a body lumen where an object is formed, the curved portion contacts the opposite area of the body lumen, and thereby pushing the cutting member 40 toward the particular area and helping the cutting member 40 in removing the object. The detail of the curved structure is described later.

The handle 20 is detachable from the catheter 10 to be reusable in multiple medical procedures. When a medical procedure is performed, the handle 20 is attached to the catheter 10 via the hub 50 so that the drive shaft 30 and the cutting member 40 can rotate according to the torque generated by the torque generating element housed in the handle 20. Thereafter, the drive shaft 30 and the cutting member 40 are inserted into a body lumen, e.g., a vein, using a guide wire. In one embodiment, the hub 50 and the handle 20 may be integrated into a single component so as not to be detachable.

Further details of the aspiration and torque generating mechanisms are described in U.S. application Ser. No. 16/998,824, the entire contents of which are incorporated by reference herein.

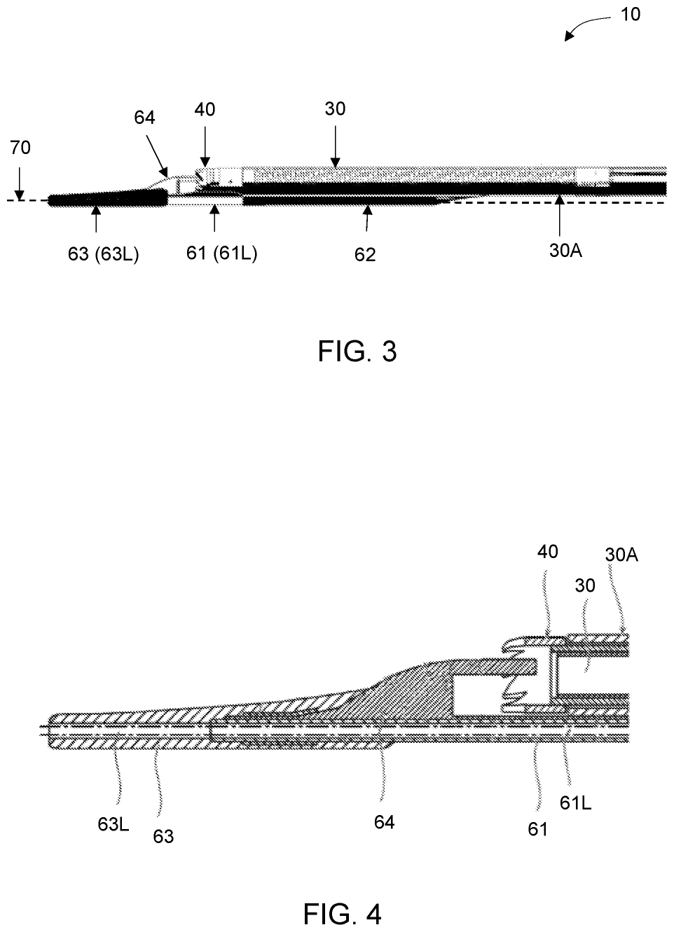

FIGS. 3 and 4 each show a distal portion of the catheter 10. The curved portion of the catheter 10 is not shown in these figures. As described above, the catheter 10 includes the cutting member 40 at the distal end thereof. The catheter 10 further includes a guide wire tube 61 having a guide wire lumen 61L, a fixing tube 62 for fixing the guide wire tube 61 to the outer tube 30A, and a distal tip 63 having a guide wire lumen 63L. The distal end of the catheter 10 is inserted into a body lumen along a guide wire 70 in a state in which the guide wire 70 passes through the guide wire lumens 61L and 63L. FIG. 4 shows the guide wire lumens 61L and 63L that form a single path for the guide wire 70 to pass. The catheter 10 and the guide wire 70 are connected to each other in a body lumen only through the guide wire lumens 61L and 63L.

For example, the guide wire tube 61 is formed of polyimides, PEEK, and the like. The fixing tube 62 is a heat-shrinkable tube that shrinks at a temperature lower than the guide wire tube 61 so that the guide wire tube 61 is strongly fixed to the outer tube 30A when heated. The distal tip 35 is a resin, preferably a thermoplastic resin, which is softer than the guide wire tube 61 so that the catheter 10 can proceed smoothly inside a body lumen.

The catheter 10 further includes a stopper 64 on the distal side of the cutting member 40, which stops the cutting member 40 from proceeding further when the catheter 10 is unintentionally guided toward the body lumen. The stopper 64 is formed of a metal material and is fixed to the guide wire tube 61. A distal portion of the stopper 64 is covered by the distal tip 63.

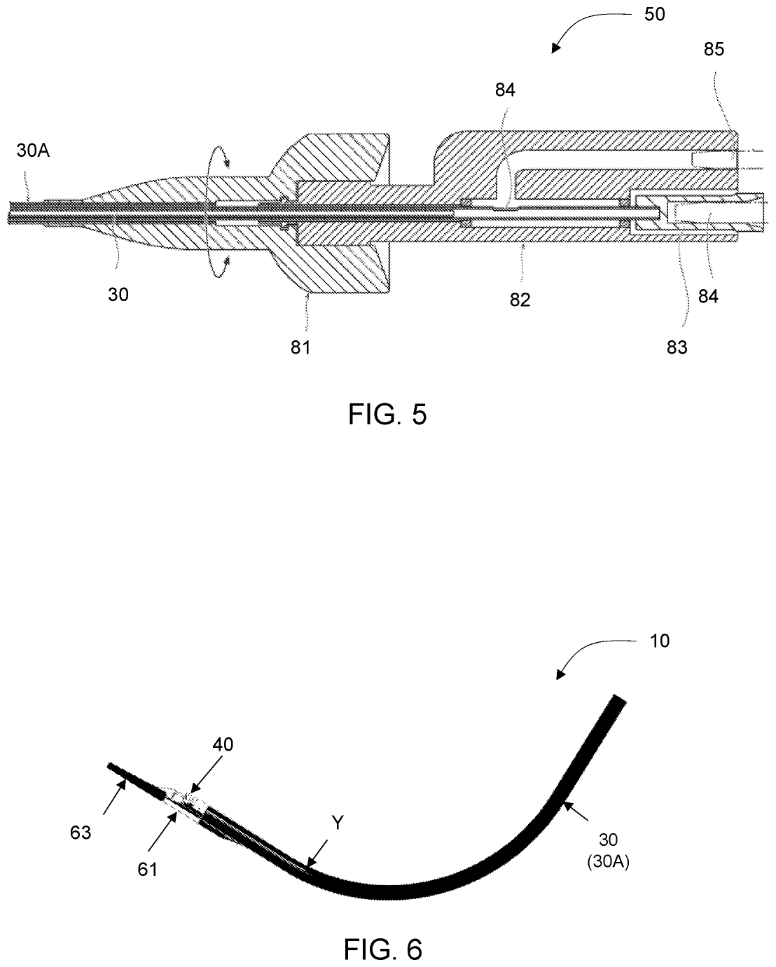

FIG. 5 depicts a proximal portion of the catheter 10 and the hub 50 into which the drive shaft 30 is inserted. The hub 50 includes a rotatable knob 81 and a connector 82 connectable to the handle 20. The knob 81 is connected to the outer tube 30A and rotatable by an operator so as to adjust an orientation and/or location of the drive shaft 30 and the cutting member 40 inside a body lumen during an operation. The knob 81 allows the outer tube 30A to rotate independently of the drive shaft 30 and the connector 82.

The drive shaft 30 is, directly or indirectly via one or more other shafts, connected to a coupler 83 with a recess 84 that can engage with a drive shaft (not shown) connected to the torque generating element inside the handle 20. Further, the aspiration lumen of the drive shaft 30 communicates with an aspiration port 85 via an opening 84 inside the connector 82. When the catheter 10 is connected to the handle 20 via the connecter 82, the generated torque is transmitted to the drive shaft 30 via the coupler 83 while the object that has entered the aspiration lumen of the drive shaft 30 is discharged from the aspiration port 85 via the opening 84.

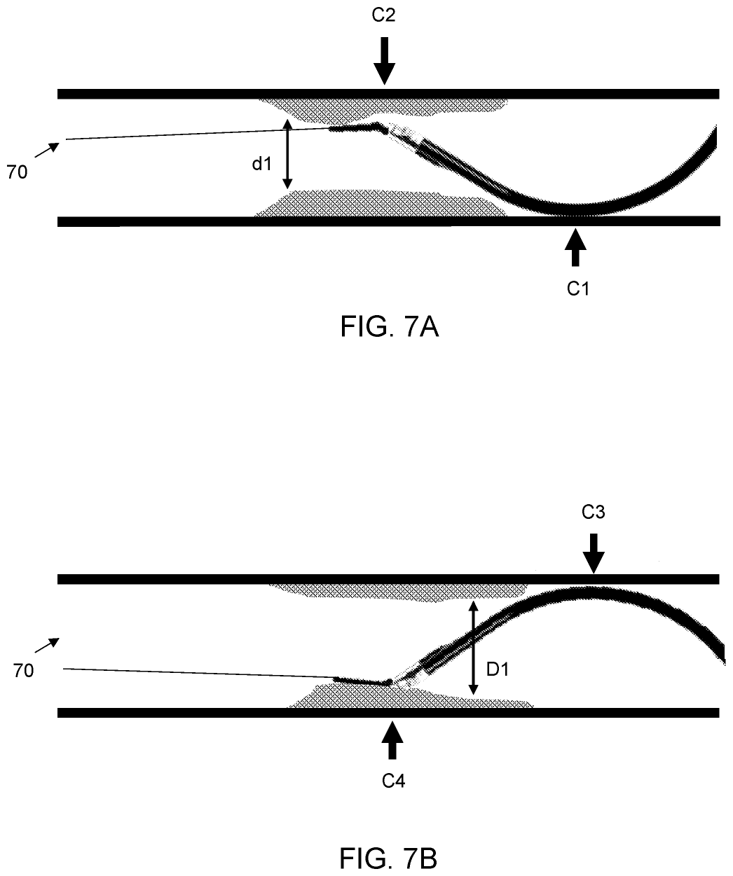

FIG. 6 depicts a curved portion of the catheter 10 in one embodiment, and FIGS. 7A and 7B depict the catheter 10 cutting an object inside a body lumen. The dimension of the elements shown in these figures is different from the actual dimension thereof. As described with reference to FIGS. 1 and 2, the catheter 10 has at least one curved portion near the distal end of the catheter 10.

As shown in FIG. 6, the catheter 10 or the drive shaft 30 has one curved portion at a position Y near the distal end of the catheter 10 or the cutting member 40 (e.g., 3-100 mm from the distal end of the catheter 10). The radius of curvature of the curved portion is, e.g., 5 mm to 60 mm. Additionally, the distal tip 63 of this embodiment has a relatively shorter length, e.g., 2 mm to 10 mm, than a length of a conventional distal tip to prevent the guide wire that is inserted in the guide wire lumen 63L at the distal tip 63 from reducing the curved bend of the drive shaft 30 too much due to the guide wire's stiffness.

When the catheter 10 is guided along a guide wire 70 inside a body lumen, its curved portion contacts an inner surface of the body lumen (e.g., at position C1 depicted in FIG. 7A), and the cutting member 40 is positioned for cutting an object formed on the opposite side of the body lumen (e.g., at position C2 depicted in FIG. 7A). The distal tip 63 is made of a soft material and thus is deformed when pushed against the body lumen. If the catheter 10 is repositioned so that its curved portion contacts the other side of the body lumen (e.g., at position C3 depicted in FIG. 7B), the cutting member 40 is positioned for cutting an object formed on the opposite side of the body lumen (e.g., at position C4 depicted in FIG. 7B). As a result, the effective cutting diameter is D1, which is larger than the cutting diameter d1 that the catheter 10 would be able to achieve without the curved portion.

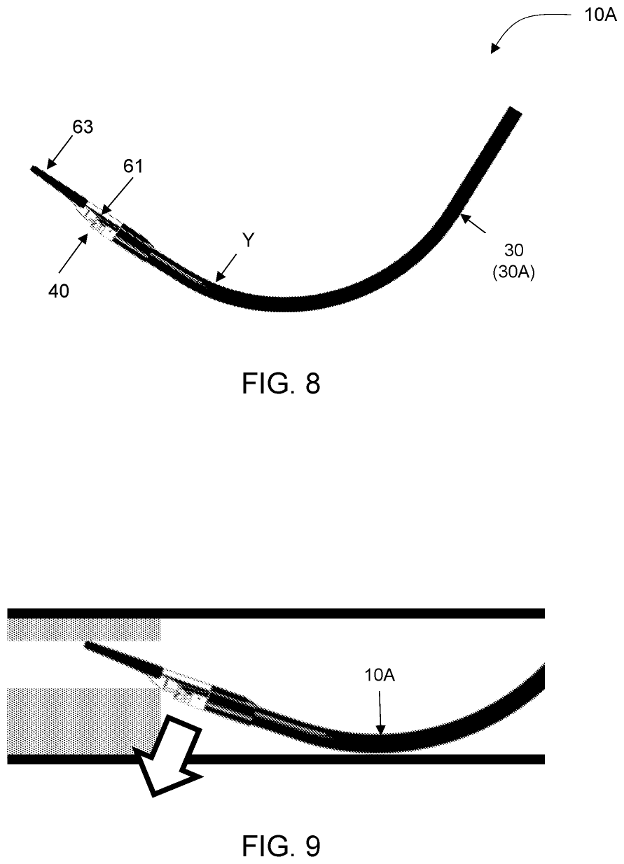

FIG. 8 depicts a catheter 10A in another embodiment. The catheter 10A has a structure similar to the catheter 10 as shown in FIG. 6, but the positions of the cutting member 40, the guide wire tube 61, and the distal tip 63 are different. As shown in FIG. 8, the cutting member 40 is located on an outer surface side of a convex part that the curved portion forms, and the guide wire tube 61 and the distal tip 63 are located on an inner surface side of the convex part. This arrangement can mitigate damage to a body lumen even when a force to move the cutting member 40 toward a surface of the body lumen is applied (see FIG. 9), because the curved portion contacts that surface first and thus the cutting member 40 contacts the body lumen from its side surface.

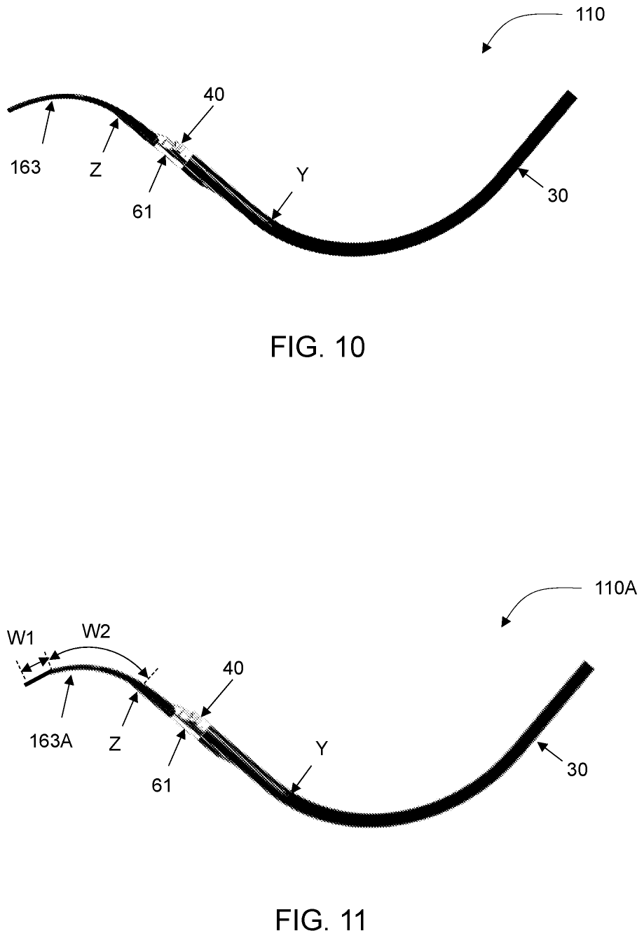

FIG. 10 depicts a catheter 110 in another embodiment with a second curved portion on a distal tip 163 at a position Z (e.g., 0-40 mm from the proximal end of the distal tip 163) in addition to the curved portion on the drive shaft 30 shown in FIG. 6. The second curved portion is curved in the opposite direction of the curvature of the drive shaft 30 with respect to the rotation axis of the cutting member 40. The radius of curvature of the second curved portion is, for example, 1 mm to 20 mm. The distal tip 163 of this embodiment also has a relatively short length, e.g., 25 mm. The second curved portion of the distal tip 163 allows for more stable positioning of the cutting member 40 with respect to the inner surfaces of the body lumen.

In yet another embodiment, as shown in FIG. 11, a catheter 110A includes a distal tip 163A with a straight portion on the distal side of the second curved portion. The length W1 of the straight portion is shorter than the length W2 of the second curved portion to enhance to enhance the guidewire trackability.

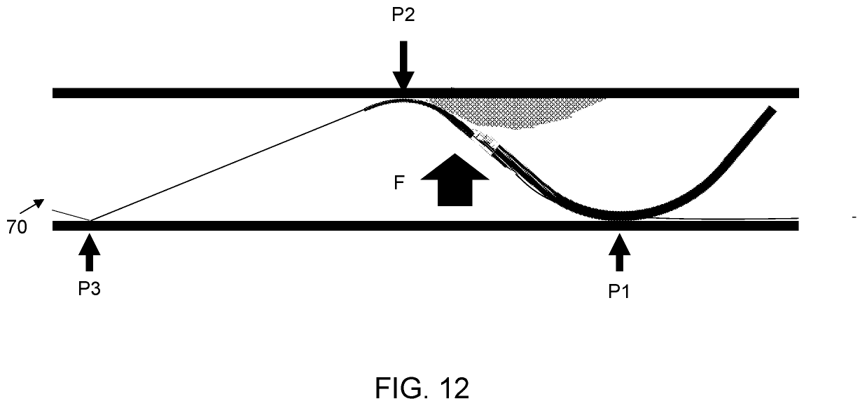

FIG. 12 schematically depicts the catheter 110 (or 110A) guided along the guide wire 70 inside a body lumen. The dimension of the elements shown in the figure is different from the actual dimension thereof. As shown in the figure, the catheter 110 with two curved portions has been guided along the guide wire 70 and has reached an object formed on a surface of the body lumen. Here, the outer tube 30A of the drive shaft 30 contacts the body lumen at point P1, the distal tip 163 contacts the body lumen at point P2, and the guide wire 70 contacts the body lumen at point P3. In this state, the cutting member 40 is supported by at least points P1 and P2 and pushed toward direction F against the object, resulting in deeper removal of the object and forming of a larger lumen inside the body lumen.

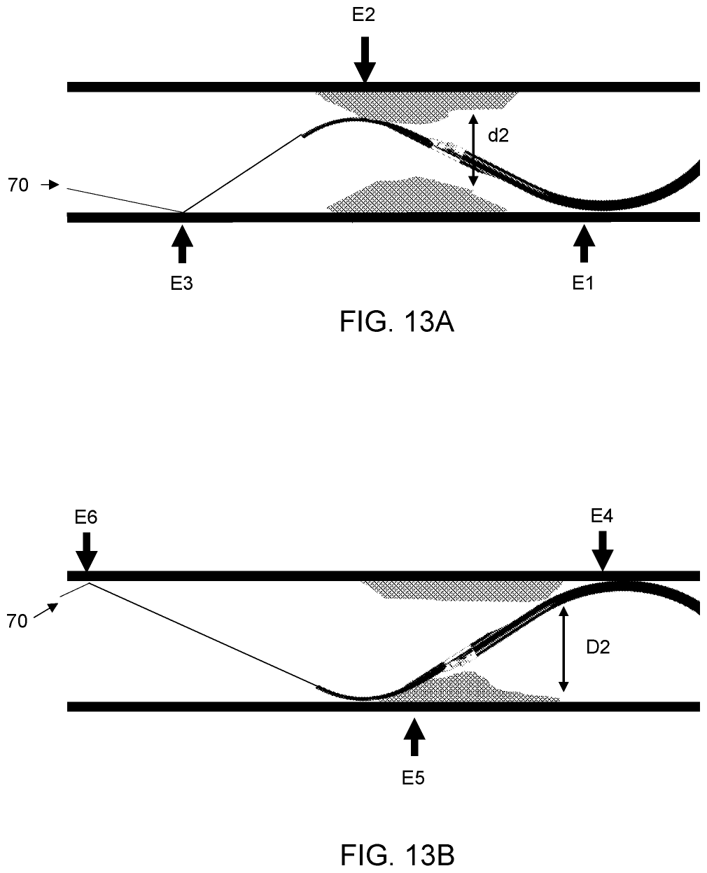

FIGS. 13A and 13B depict that catheter 110 (or 110A) cutting an object inside a body lumen. The dimension of the elements shown in these figures is different from the actual dimension thereof. As described with reference to FIG. 12, when the catheter 110 is guided along the guide wire 70 inside a body lumen, each curved portion contacts an inner surface of the body lumen (e.g., at positions E1 and E2 depicted in FIG. 13A), and the cutting member 40 is positioned for cutting an object formed on the inner surface (e.g., at position E2). The guide wire contacts the body lumen at position E3. If the catheter 110 is repositioned so that each curved portion contacts the other side of the body lumen (e.g., at positions E4 and E5 depicted in FIG. 13B), the cutting member 40 is positioned for cutting an object formed on the opposite side of the body lumen (e.g., at position E5). The guide wire contacts the body lumen at position E6. As a result, the effective cutting diameter is D2, which is larger than the cutting diameter d2 that the catheter 10 would be able to achieve without the curved portion.

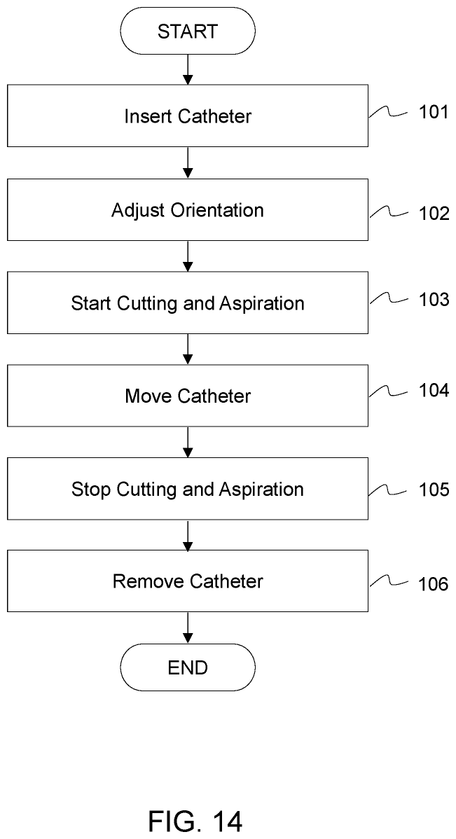

FIG. 14 depicts a flowchart of a method for removing an object in a body lumen using the medical device 1 or the catheter 10 described above. At the beginning of a medical operation, the operator connects the catheter 10 to the handle 20 via the hub 50 and inserts the guide wire 70 into the body lumen from its distal end using an introducer sheath so that the guide wire 70 passes near a target location where the object is formed. After those steps, the operator passes the proximal end of the guide wire 70 through the guide wire lumens 61L and 63L of the catheter 10, and then inserts the catheter 10 into the body lumen along the guide wire 70 (Step 101).

Once the distal end of the catheter 10 has reached the target location, the operator rotates the knob 81 to adjust the orientation and/or location of the catheter 10 inside the body lumen so that the cutting member 40 is directed toward the object by the curvature (Step 102). The operator then turns on the aspiration and torque generating mechanisms of the handle 20 to start cutting and aspirating the object (Step 103), and moves the catheter 10 forward along the guide wire 70 until the target object is removed and aspirated (Step 104).

Once the target object has been removed, the operator turns off the aspiration and torque generating mechanisms (Step 105), and removes the catheter 10 from the body lumen (Step 106). The guide wire and introducer sheath are removed thereafter.

The order of one or more of the steps shown in FIG. 14 can be modified. For example, the aspiration and torque generating mechanisms can be turned on at any time after the catheter 10 has been inserted into the body lumen. Additionally, one or more of the steps can be repeated if necessary. For example, the orientation of the catheter 10 can be adjusted at any time during the operation to remove the object.

What is claimed is:

1. A catheter for removing an object in a body lumen, comprising:
an outer tube;
a rotatable drive shaft surrounded by the outer tube;
a cutting member connected to a distal end of the drive shaft to be rotated by the drive shaft with respect to a rotation axis and by which the object is cut;
a guide wire tube attached to a distal end of the outer tube and having a first guide wire lumen extending adjacent to the cutting member along the rotation axis; and
a distal tip attached to a distal end of the guide wire tube and having a second guide wire lumen extending parallel to the rotation axis and communicating with the first guide wire lumen, wherein
the drive shaft includes a first curved portion near the distal end of the drive shaft to cause the distal tip to contact a first inner surface of the body lumen when the first curved portion contacts a second inner surface of the body lumen that is on an opposite side of the body lumen with respect to the first inner surface.

2. The catheter according to claim 1, wherein the distal tip includes a second curved portion that is curved to cause a distal end of the distal tip to be directed toward a center of the body lumen when the distal tip contacts the first inner surface of the body lumen and the first curved portion contacts the second inner surface of the body lumen.

3. The catheter according to claim 2, wherein a radius of curvature of the first curved portion is greater than a radius of curvature of the second curved portion.

4. The catheter according to claim 3, wherein the radius of curvature of the first curved portion is 5 mm to 60 mm.

5. The catheter according to claim 3, wherein the radius of curvature of the second curved portion is 1 mm to 20 mm.

6. The catheter according to claim 2, wherein the second curved portion is located at a distance of 0 mm to 40 mm from a proximal end of the distal tip.

7. The catheter according to claim 1, wherein the first curved portion is located at a distance of 3 mm to 100 mm from the distal end of the distal tip.

8. The catheter according to claim 1, wherein
the first curved portion is curved in a first direction when viewed from the cutting member, and
the distal tip and the rotation axis of the cutting member is arranged in this order in the first direction.

9. The catheter according to claim 1, wherein the first guide wire lumen is straight, and the second guide wire lumen is at least partially curved.

10. The catheter according to claim 1, wherein a proximal end of the first guide wire lumen is between the distal end of the outer tube and the first curved portion.

11. A medical device for removing an object in a body lumen, comprising:
a handle including a motor configured to generate rotation torque; and
a catheter connectable to the handle and including:
an outer tube;
a drive shaft surrounded by the outer tube and rotatable by the generated rotation torque;
a cutting member connected to a distal end of the drive shaft to be rotated by the drive shaft with respect to a rotation axis and by which the object is cut;
a guide wire tube attached to a distal end of the outer tube and having a first guide wire lumen extending adjacent to the cutting member along the rotation axis; and
a distal tip attached to a distal end of the guide wire tube and having a second guide wire lumen extending parallel to the rotation axis and communicating with the first guide wire lumen, wherein
the drive shaft includes a first curved portion near the distal end of the drive shaft to cause the distal tip to contact a first inner surface of the body lumen when the first curved portion contacts a second inner surface of the body lumen that is on an opposite side of the body lumen with respect to the first inner surface.

12. The medical device according to claim 11, wherein
the catheter is connectable to the handle via a hub, and
the hub includes a knob connected to and rotatable with the outer tube.

13. The medical device according to claim 11, wherein the distal tip includes a second curved portion that is curved to cause a distal end of the distal tip to be directed toward a center of the body lumen when the distal tip contacts the first inner surface of the body lumen and the first curved portion contacts the second inner surface of the body lumen.

14. The medical device according to claim 13, wherein a radius of curvature of the first curved portion is greater than a radius of curvature of the second curved portion.

15. The medical device according to claim 14, wherein the radius of curvature of the first curved portion is 5 mm to 60 mm.

16. The medical device according to claim 14, wherein the radius of curvature of the second curved portion is 1 mm to 20 mm.

17. The medical device according to claim 13, wherein the second curved portion is located at a distance of 0 mm to 40 mm from a proximal end of the distal tip.

18. The medical device according to claim 11, wherein the first curved portion is located at a distance of 3 mm to 100 mm from the distal end of the distal tip.

19. The medical device according to claim 11, wherein
the first curved portion is curved in a first direction when viewed from the cutting member, and
the distal tip and the rotation axis of the cutting member is arranged in this order in the first direction.

20. A method for removing an object in a body lumen using a catheter that includes:
an outer tube,
a rotatable drive shaft surrounded by the outer tube,
a cutting member connected to a distal end of the drive shaft to be rotated by the drive shaft with respect to a rotation axis and by which the object is cut,
a guide wire tube attached to a distal end of the outer tube and having a first guide wire lumen extending adjacent to the cutting member along the rotation axis, and
a distal tip attached to a distal end of the guide wire tube and having a second guide wire lumen extending parallel to the rotation axis and communicating with the first guide wire lumen, wherein
the drive shaft includes a first curved portion near the distal end of the drive shaft, the method comprising:
inserting the catheter into the body lumen;
adjusting an orientation of the catheter in the body lumen so that the distal tip contacts a first inner surface of the body lumen when the first curved portion contacts a second inner surface of the body lumen that is on an opposite side of the body lumen with respect to the first inner surface;
moving the catheter forward and removing the object; and
removing the catheter from the body lumen.
